(19) United States
(12) Patent Application Publication
(10) Pub. No.: US 2004/0002135 A1
(43) Pub. Date: Jan. 1, 2004

(54) Adapted NS0 cell lines with the ability to grow under glutamine-free conditions

(76) Inventors: Paul W. Sauer, Fremont, CA (US); John E. Burky, Newark, CA (US)

Correspondence Address: HOWREY SIMON ARNOLD & WHITE, LLP, BOX 34, 301 RAVENSWOOD AVE., MENLO PARK, CA, 94025, US

(21) Appl. No.: 10/386,200

(22) Filed: Mar. 10, 2003

Related U.S. Application Data
(60) Provisional application No. 60/368,552, filed on Mar. 28, 2002.

(51) Int. Cl. C12P 21/02; C12N 5/08; C07K 16/18; C12N 5/06

(52) U.S. Cl. 435/69.1; 435/326; 435/366; 435/320.1; 530/387.1

(57) ABSTRACT

The present invention provides for an adapted NS0 myeloma cell line that has the ability to grow in glutamine-deficient or glutamine-free condition. In one particular embodiment, the adapted NS0 cell line has an Accession Number ATCC No. PTA-4106.

This application claims the benefit of priority of the U.S. provisional application U.S.S No. 60/368,552 filed Mar. 28, 2002, which is incorporated by reference in its entirety.

### TECHNICAL FIELD

This invention relates to the field of cell culture technology. In particular, it relates to a NS0 mycloma cell line that has the ability to grow in glutamine-free cell culture media, and thereby reduce ammonia toxicity associated with glutamine metabolism. In one example, the adapted NS0 cell line has an Accession Number ATCC No. PTA-4106.

### BACKGROUND OF THE INVENTION

Ammonia is known to be a toxic byproduct of glutamine metabolism. Its accumulation has adverse effect on cell culture performance such as reduced growth rates, changes in metabolic rates, perturbation of protein processing, and virus replication. Ammonia can be produced during cell culture by two separate pathways involving glutamine glutaminolysis (metabolism) and glutamine deamidation (chemical breakdown). Therefore, glutamine provides the main source of ammonia. (Chen, et al., Chin. J. Biotechnol. 8(4): 255-61 (1992)). Accordingly, eliminating glutamine entirely from the cell culture media would significantly reduce ammonia production.

In glutamine-free medium, cells need to biosynthetically produce their own glutamine by way of the glutamine synthetase (GS) pathway. The levels of GS vary in different tissues and in many cell types. (Labow, et al., Am. J. Physiol. 276:E1136-1145 (1999)). Consequently, a number of established cell lines derived from the tissues other than myeloma or hybridoma can be adapted to grow in glutamine-free media, often after a period of adaptation in reduced levels of glutamine. However, all myeloma and hybridoma cell lines analyzed have an absolute requirement for glutamine presumably due to insufficient glutamine synthetase activity (Bebbington, C. R., et al., Biotechnology, vol. 10, pp 169-175 (1992); Barnes, L. M., et al., Biotechnology and Bioengineering, Vol. 73: pp 261-270 (2001)).

As a result of this finding, researchers have used different methods in order to take advantage of the glutamine synthetase gene. Bebbington et al. employed a strategy exploiting this reaction. They transfected NS0 cells with the GS-gene and used the GS-gene as a selectable and amplifiable marker for isolating transfectants, since normal NS0 cells are incapable of growing without supplemental glutamine (Bebbington et al.). This transfection process has also been disclosed in International Patent Application WO87/04462.

The same recombinant DNA technique has been utilized for the purpose of developing glutamine-independent cell lines to overcome the problem of toxic ammonia accumulation. International Patent Applications WO86/05807 and WO89/10404 disclose the establishment of glutamine-independent cell lines by transforming lymphoid cells with vectors comprising a gene encoding GS. However, these references do not disclose the method of gradual adaptation provided in the present invention for isolating a glutamine-independent NS0 cell line.

Successful production of glutamine-independent cell lines by adaptation has been reported in kidney cells, such as McCoy cell line and MDCK cell line (McDermott and Butler, J. Cell Sci. 104:51-58 (1993)). Hassell and Butler also disclose methods for the adaptation of three animal cell lines to media in which glutamine is replaced with either glutamate or 2-oxoglutarate (Hassell and Butler, J. Cell Sci. 96(Part 3):501-8 (1990)). In one study, BHK-21 cells have been shown to grow in a less ammoniagenic medium (Christie and Butler, Biotechnol. Bioeng. 64(3):298-309 (1999)). Higher frequency of spontaneous development of glutamine-independent variants has also been obtained by treating the V79-56 cells, a subline of Chinese hamster cells, with the mutagen ethyl methane sulfonate or by exposure to 5-azacytidine and sodium butyrate, although V79-56 cells have an absolute requirement for exogenous glutamine (Harris, Somatic Cell and Molecular Generics 10(3): 275-281 (1984)). However, these references do not disclose the development of a glutamine-independent NS0 cell line. By successfully producing a glutamine-independent NS0 cell line, the present invention has solved a long-lasting technical problem, that is, the inability to grow NS0 cells without prior adaptation in the absence of glutamine due to their absolute requirement for glutamine (Barnes, L. M. et al.)

Glutamine independence has been developed in mouse NS1 myeloma cells by growing in glutamine limited chemostat culture supplemented with glutamate. In the chemostat, a steady state was achieved during which glutamine was essentially exhausted. After about 55 days, the viable cell concentration increased, the residual glutamate concentration in the bio-reactor decreased, and a glutamine independent subpopulation had been selected. (Birch, et al., Cytotechnology 15:11-16 (1994)).

The present invention has a number of novel features in view of Birch, et al.. A glutamine-independent NS0 cell line has been developed in the present invention for the first time. NS0 cells are very different from NS1 cells since NS1 cells express intracellular light chain while NS0 cells do not. In addition, NS0 cells are much more commonly used than NS1 cells in the industry for the purpose of producing recombinant antibodies. Therefore, the glutamine-independent NS0 cell line is of great significance as a foundation for cell culture work in biomedical industry. Moreover, the present invention employs adaptation techniques, wherein cells were maintained in stationary flasks and passaged using gradually reduced glutamine concentrations. In contrast, Birch et al. employed chemostat method by selecting cells in a suspended culture in a bioreactor (Birch, et al.).

The present invention provides an adapted NS0 myeloma cell line (having Accession ATCC No. PTA-4106), which has the ability to grow in a glutamine-independent medium. The deposit of this adapted NS0 mycloma cell line (PTA-4106) was received by American Type Culture Collection (ATCC), P. O. Box 1549, Manassas, Va. 20108 on Feb. 27, 2002. The NS0 cell line, as obtained from the ECACC, is also serum- and cholesterol- independent. Previously, the NS0 cell line was made to be adapted to serum-free and cholesterol-free medium by the scientists employed by Protein Design Labs, Inc.

### SUMMARY OF THE INVENTION

The present invention is directed to a cell line capable of growing in a medium containing less than about 5 mM, 3 mM, 1 mM, 0.4 mM, 0.3 mM, or containing about 0 mM glutamine. Preferably such a cell line is an adapted NS0 mycloma cell line.

The present invention is directed to a cell derived from a NS0 myeloma cell line capable of growing in a glutamine-free medium, preferably identified by the accession number ATCC No. PTA-4106.

The present invention is further directed to a NS0 myeloma cell line having the accession number ATCC No. PTA-4106.

The present invention is further directed to a method of reducing toxicity of a cell culture comprising growing the NS0 mycloma cell line that is capable of growing in a glutamine-free medium in a glutamine-free medium in said cell culture.

The present invention is further directed to a method of making a recombinant polypeptide comprising: a) transfecting an expression vector comprising of a DNA molecule encoding the recombinant polypeptide into a cell of a NS0 mycloma cell line capable of growing in a glutamine-free medium; b) culturing said transformed cell in a glutamine-free medium; and c) secreting said recombinant polypeptide.

The present invention is further directed to a method of generating a NS0 myeloma cell line capable of growing in a glutamine-free medium comprising a) culturing a NS0 cell line in a medium supplied with glutamine; b) reducing concentration of glutamine gradually until the cell culture medium contains no glutamine; and c) selecting NS0 cells that survive in the cell culture medium containing no glutamine.

### BRIEF DESCRIPTION OF THE FIGURES

FIG. 1. Adaptation of the NS0 cell line to glutamine-free media. The NS0 cell line was cultured initially in PFBM-1 containing 5.0 mM glutamine. After gradually reducing the glutamine concentration to 0.4 mM, the cell population showed a significant decrease in viability. After viability recovered, glutamine concentration reduction continued. At 0.3 mM glutamine the cell population exhibited a prolonged and significant decline in viability. After recovery (approximately day 75) the population was subjected to glutamine-free medium (0 mM) and a relatively minor decrease in cell viability was observed. A glutamine-free population of NS0 cells was isolated after 98 days of culture.

FIG. 2. Adaptation of NS0 cell line to glutamine-free medium. The NS0 cell line was cultured initially in PFBM-1 containing 5 mM glutamine (see Examples for details). The glutamine concentration was reduced over a 30 day period from 5 mM to 0.4 mM by a series of sequential dilutions (i. e., 1:1, 1:2, 1:3 . . . ) of 100% PFBM-1 to a 1:12 dilution of PFBM-1 to glutamine-free PFBM-1. A significant decrease in viability was observed once the glutamine concentration reached 0.4 mM. After viability recovered, glutamine concentration reduction resumed. At 0.3 mM glutamine the cell population exhibited a prolonged and significant decline in viability. After recovery (approximately day 75) the population was subjected to glutamine-free medium (0 mM) and a relatively minor decrease in cell viability was observed. A glutamine-independent population of NS0 cells was isolated after 98 days of adaptation.

FIGS. 3 and 4. A viable cell density for fed-batch cultures of the glutamine-independent NS0 cells cultured in 15L bioreactors. For runs 1A and 1B the supply of oxygen was exhausted. It is unknown what the peak viable cell density would have reached had the oxygen not been exhausted.

FIG. 5. Viable cell density profile of glutamine-independent NS0 cells producing a humanized IgG1 antibody.

FIG. 6. The antibody titer profile of two fed-batch fermentations of a humanized antibody producing cell line. Cultures were fed with different amounts of cystine in the feed medium.

### DETAILED DESCRIPTION OF THE INVENTION

The present invention is directed to a cell line capable of growing in a medium containing less than about 5 mM, 3 mM, 1 mM, 0.4 mM, 0.3 mM, or 0 mM glutamine. Preferably such a cell line is an adapted NS0 myeloma cell line.

In a preferred embodiment, the NS0 myeloma cell line is also capable of growing in a serum or/and cholesterol-free condition (the cell culture medium contains no serum or/and cholesterol).

The present invention provides for a cell derived from the NS0 myeloma cell lines described herein, preferably identified by the accession number ATCC No. PTA-4106. A cell derived from the NS0 myeloma cell line means a cell descending from or obtained from said cell line.

The present invention provides for an adapted NS0 myeloma cell line with Accession Number ATCC No. PTA-4106, that has the ability to grow in glutamine-deficient or glutamine-free condition. Said NS0 cell line is able to grow in a cell culture medium that has 0 mM of glutamine (glutamine-free culture medium). Such a glutamine-free culture medium includes, but is not limited to, PFBM-1/GS, which comprises glutamine-free PFBM-1 supplemented with 1×GS supplement (Cat. No. 58672-77P, JRH Biosciences). The concentrations of components in 50×GS are disclosed in Example 1. Said NS0 cell line can also grow in glutamine-deficit condition, such as in a cell culture medium containing less than 5 mM of glutamine.

The present invention also provides for a method of using said adapted NS0 myeloma cell line to provide cell culture conditions of reduced toxicity, wherein said cell line has the ability to grow in glutamine-deficit or glutamine-free condition. In one aspect, the method comprises: growing said adapted NS0 myeloma cell line in a glutamine-deficient or glutamine-free medium, or said adapted NS0 cell line can be grown in a glutamine-free medium, including but not limited to, PFBM-1/GS as disclosed in Example 1. In another aspect, the method comprises: growing said adapted NS0 myeloma cell line in a serum-free, cholesterol-free and glutamine-free medium. Such a medium includes, but is not limited to, PFBM-1/GS medium as disclosed in Example 1.

The adapted NS0 cell line can be used as a host for transfection of desired foreign genes and production of desired polypeptides such as antibodies. It is established that eliminating glutamine entirely from the cell culture media would significantly reduce ammonia production. Thereby, said glutamine-independent NS0 cell line produces little to no ammonia during the course of culturing. As a result, the integral of viable cell concentration increases. So does the rate of production of the desired polypeptide by the NS0 cell line.

The present invention is further directed to a method of making a recombinant polypeptide comprising: a) transfecting an expression vector comprising a DNA molecule encoding the recombinant polypeptide into a cell of a NS0 cell line capable of growing in a glutamine-free medium; b) culturing said transformed cell in a glutamine free medium; and c) secreting said recombinant polypeptide. Preferably, said recombinant polypeptide is a recombinant antibody, and more preferably, a humanized antibody. The DNA molecules encoding the recombinant polypeptides are linked operably with expression vectors via standard molecular cloning techniques known in the art. Methods of transfecting expression vectors into host cells, such as the adapted NS0 cells, are also known in the art.

The produced polypeptides have great therapeutic value to human disease treatment and prevention. For instance, the antibodies can be used for the prevention of organ transplant rejection, for the treatment of breast cancer, respiratory syncytial virus disease and acute myeloid leukemia. It can also be used for the treatment of various types of cancers including solid tumors, leukemias and lymphomas. It may also be used for the treatment of inflammatory conditions such as asthma, allergy and trauma-induced disorders autoimmune diseases including psoriasis, multiple sclerosis, rheumatoid arthritis, inflammatory bowel disease, Type I diabetes, Crohn's disease, and cardiovascular diseases. The adapted NS0 cell lines are useful in efficient production of such therapeutic proteins or antibodies for the treatment of human diseases.

The present invention also provides for a method of generating a NS0 myeloma cell line capable of growing in a glutamine-free condition. Said method comprises gradual adaptation of a NS0 cell line to glutamine-free condition. Said method further comprises: culturing a NS0 cell line in a cell culture medium supplied with glutamine; reducing the concentration of glutamine gradually until the cell culture medium contains no glutamine; and selecting the surviving population (the NS0 cells that survive the glutamine-free condition). NS0 cells are maintained in stationary flasks and passage using gradually reduced glutamine concentrations over a period of several months, for example, 1, 3, or 5 months. The glutamine concentration can be gradually reduced in various ways as long as the sudden decrease of glutamine concentration is avoided. In a preferred embodiment, glutamine concentration was reduced over about a 20, 30, or 50 day period from about 5 mM to about 0.4 mM via series of sequential dilutions and then the concentration further slowly reduced to 0 mM over the next about 30, 50, 60, 80, or 100 days. The experimental details of this method are disclosed in Example 1.

### EXAMPLES

### Example 1

Materials and Methods

The experimental objective was the gradual adaptation of the NS0 host cell line to glutamine-free conditions. The medium used was glutamine-free PFBM-1 supplemented with 1×GS Supplement (Cat. No. 58672-77P, JRH Biosciences). This GS-supplemented medium will henceforth be referred to as PFBM-1/GS. The concentration of the components in the neat 50×JRH GS Supplement is presented in Table 1. The PFBM-1/GS medium was supplemented with the desired concentration of glutamine. The initial concentration of glutamine in the PFBM-1/GS medium was 5.0 mM.

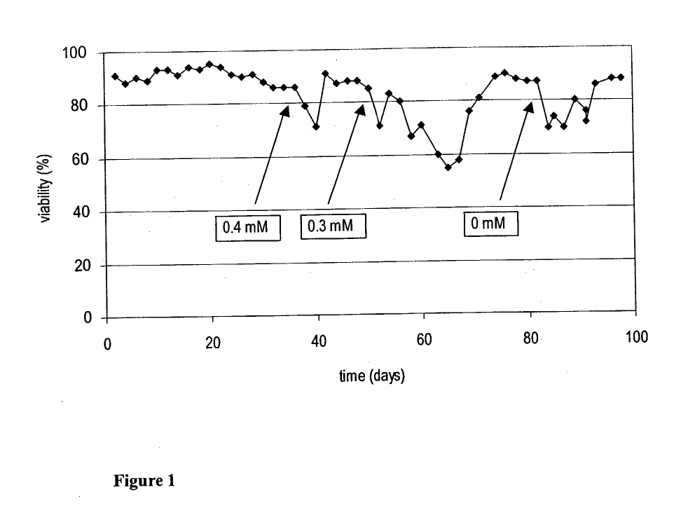

The NS0 cell line used in this experiment was derived from the serum-independent and cholesterol-independent NS0 cell line developed previously by Protein Design Labs, Inc. One vial of these frozen NS0 cells was thawed into PFBM-1/GS medium. The cells were expanded over a period of days in a T-175 flask and were then slowly adapted to glutamine-free conditions by slowly reducing the concentration of glutamine in PFBM-1/GS basal medium over a period of three months as shown in FIG. 1.

TABLE 1
Concentration of components in 50x JRH GS Supplement. The 50x
stock solution is added to PFBM-1 such that the final concentration
is 1x - this medium is designated PFBM-1/GS.

| 50x JRH GS Supplement | Concentration (mg/L) |
|---|---|
| L-Alanine | 450 |
| L-Asparagine | 3,750 |
| L-Aspartic Acid | 650 |
| L-Glutamic Acid | 3,750 |
| L-Proline | 575 |
| L-Serine | 500 |
| Adenosine | 350 |
| Guanosine | 350 |
| Cytidine | 350 |
| Uridine | 350 |
| Thymidine | 12 |

Results

The viability profile of the NS0 cells during the adaptation period is presented in FIG. 1. Declines in viability can be observed after the glutamine concentration was lowered to 0.4, 0.3 and 0.0 mM. During these declines in viability a sub-population of glutamine-independent NS0 cells arose. After three months of weaning, the cells were completely glutamine-independent.

The purpose of this work was to develop a glutamine-independent NS0 cell line that could be used as a host cell line for transfections. In an attempt to overcome glutamine dependence, the NS0 cell line was slowly adapted to glutamine-free conditions. The NS0 variant no longer requires supplemental glutamine for growth. A glutamine-independent cell line, which produces little to no ammonia during the course of a culture, holds great promise as a foundation for future cell line development work.

### Example 2

This study reports the isolation of a glutamine independent NS0 cell line. Creation of a glutamine independent NS0 cell line will enable the use of glutamine-free medium which will substantially reduce the production of ammonia, a known toxic metabolite of cell culture. The approach used herein was slow adaptation of NS0 cells to glutamine-free medium conditions (i.e. isolate a glutamine independent cell population). Our studies demonstrated proof of concept (run glutamine independent NS0 cell line in 10L bioreactors monitoring ammonia concentration), as well as the isolated cell line is an appropriate host for recombinant humanized antibody expression.

Materials and Methods

A serum-free, protein-free medium similar to Hybridoma SFM (invitrogen, Cat. No. 12045-084) was used. The medium was formulated without glutamine and supplemented with 1×GS Supplement (JRH Biosciences, Cat. No. 58672-77P). The glutamine-free medium was mixed with normal medium, containing 5 mM glutamine, in decreasing ratios until the cells were adapted to a completely glutamine-free environment. Ammonia concentration was quantified using a colorimetric assay Kodak Biolyzer (Raritan, N.J.). Glutamine and glutamate concentrations were measured using a YSI 2700 Select (Yellow Springs, Ohio). Cell counting was performed manually on a hemacytometer using the trypan blue dye exclusion method for all data except bioreactor runs 3A and 3B which were performed using the Cedex automated cell counter (Innovatis, Germany). Fed-batch bioreactor runs were performed in two 15L Biostat Eds (B. Braun Biotech) controlled by separate DCUs.

Results

Adaptation Results

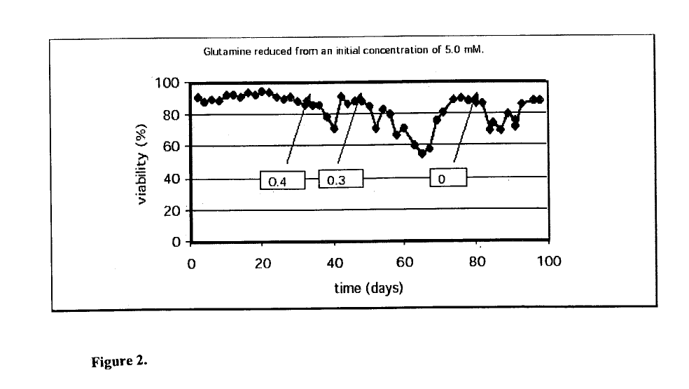

The viability profile of the NS0 cells during the adaptation period was presented in FIG. 2. Declines in viability were observed after the glutamine concentration was lowered to 0.4, 0.3 and 0.0 mM. During these declines in viability a sub-population of glutamine-independent NS0 cells arose. After three months of weaning, the cells were completely glutamine-independent. The NS0 cell line was cultured initially in PFBM-1 containing 5 mM glutamine (see Materials and Methods for details). The glutamine concentration was reduced over a 30 day period from 5 mM to 0.4 mM by a series of sequential dilutions (i. e., 1:1, 1:2, 1:3 . . . ) of 100% PFBM-1 to a 1:12 dilution of PFBM-1 to glutamine-free PFBM-1. A significant decrease in viability was observed once the glutamine concentration reached 0.4 mM. After viability recovered, glutamine concentration reduction resumed. At 0.3 mM glutamine the cell population exhibited a prolonged and significant decline in viability. After recovery (approximately day 75) the population was subjected to glutamine-free medium (0 mM) and a relatively minor decrease in cell viability was observed. A glutamine-independent population of NS0 cells was isolated after 98 days of adaptation.

Bioreactor Results

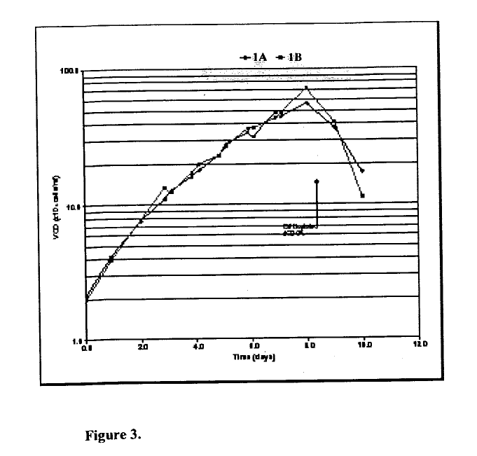

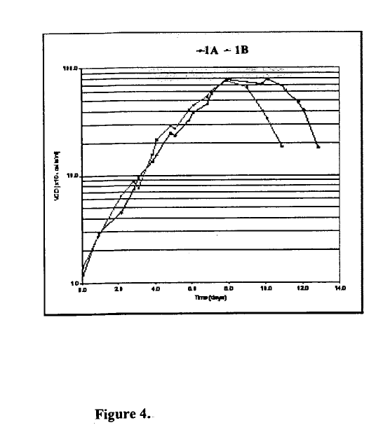

Two sets of 2×15L bioreactors were run to examine cell culture performance and ammonia productivity of the newly isolated glutamine-independent NS0 call line. FIG. 3 and FIG. 4 illustrated viable cell density profiles for cells cultured in glutamine-free conditions. Table 2 provided relevant cell culture performance data including final ammonia concentrations. Ammonia was undetectable for runs 1A, 1B, 2A and 2B.

TABLE 2
The data for Runs 1A and 1B extending through day 8.0. Data obtained
after the cultures ran out of oxygen were excluded from the summary.
Ammonia concentrations were below the detection limit of the assay
for four of the six runs. The reference culture is an NS0 cell line
expressing the same humanized antibody as runs 3A and 3B.

| | Fed-Batch Data | | | | | | |
|---|---|---|---|---|---|---|---|
| | | | | | | | Reference |
| | 1A | 1B | 2A | 2B | 3A | 3B | Culture |
| run time (days): | 8.0 | 9.0 | 12.9 | 10.9 | 11.9 | 12.8 | 12.8 |
| final working volume (L): | 8.2 | 9.8 | 7.6 | 9.4 | 10.3 | 10.0 | 9.5 |
| final antibody conc. (mg/L): | N/A | N/A | N/A | N/A | 701 | 972 | 866 |
| integral of viable cell conc. | 15 | 15 | 41 | 29 | 36 | 40 | 33 |
| (10³ cells day/L): | | | | | | | |
| glutamine consumed (mmol/L): | N/A | N/A | N/A | N/A | N/A | N/A | 56 |
| glucose consumed (g/L): | 5.5 | 7.9 | 10.3 | 8.7 | 12.4 | 11.7 | 11.0 |
| lactate produced (g/L): | 2.5 | 5.1 | 3.5 | 3.3 | 5.9 | 3.2 | 1.7 |
| specific glutamine consumption | N/A | N/A | N/A | N/A | N/A | N/A | 1.7 |
| rate (mmol/10³ cells day): | | | | | | | |
| specific glucose consumption | 0.37 | 0.52 | 0.25 | 0.30 | 0.34 | 0.29 | 0.34 |
| rate (g/10³ cells day): | | | | | | | |
| specific lactate production rate | 0.16 | 0.33 | 0.09 | 0.11 | 0.16 | 0.08 | 0.05 |
| (g/10³ cells day): | | | | | | | |
| final osmolaity (mOsm/kg H₂O): | 330 | 376 | 375 | 408 | 410 | 370 | 482 |
| final lactate conc. (g/L): | 2.5 | 5.1 | 3.6 | 3.3 | 5.9 | 3.3 | 1.8 |
| final ammonia conc. (mM): | n.d. | n.d. | n.d. | n.d. | 2.4 | 1.9 | 13.6 |
| peak viable cell conc. | 5.6 | 7.2 | 7.8 | 7.6 | 7.5 | 7.0 | 6.1 |
| (10³ cells/mL): | | | | | | | |

Viable Cell Density of a Glutamine Independent NS0 Cell Line

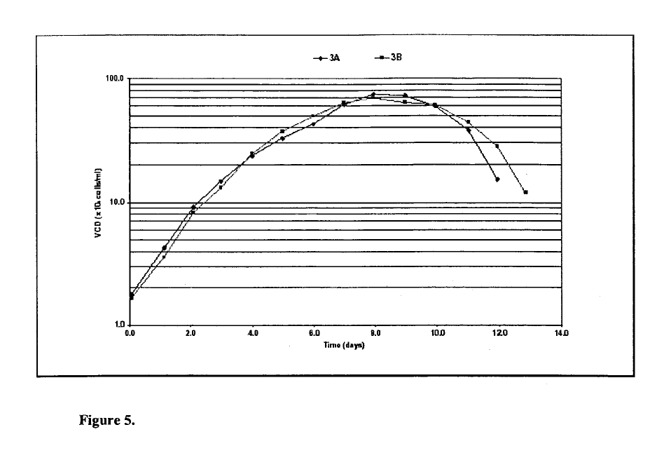

FIGS. 3 and 4 showed a viable cell density for fed-batch cultures of the glutamine-independent NS0 cells cultured in 15L bioreactors. For runs 1A and 1B the supply of oxygen was exhausted. The results indicated that produced glutamine-independent NS0 cells are able to grow in the glutamine-free medium at a desired density. FIG. 5 also showed the viable cell density profile of glutamine-independent NS0 cells producing a humanized IgG1 antibody.

Bioreactor Data from a Glutamine-Independent IgG1 Producing Transfectant

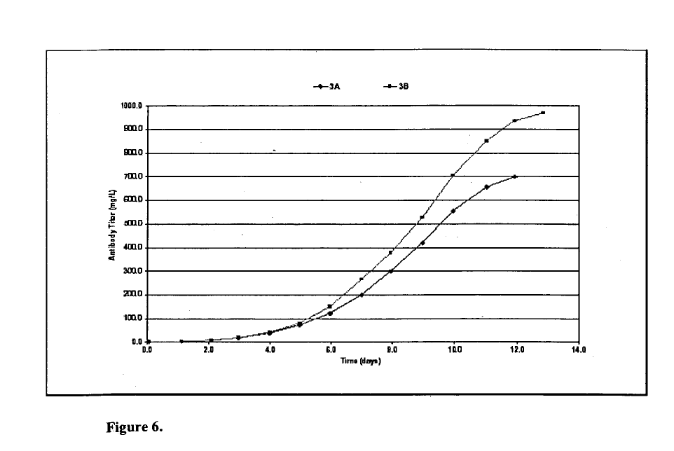

Cells from a primary transfectant were run in two 15L bioreactors to assess cell culture performance and antibody productivity. FIG. 6 illustrated the viable cell density profile of the two bioreactor runs. Table 2 showed the relevant cell culture performance data. Ammonia was detectable at the end of the run but at a much lower level than a run with glutamine.

Antibody Production of a Glutamine-Independent IgG1 Producing Transfectant

FIG. 6 showed the antibody titer profile of two fed-batch fermentations of a humanized antibody producing cell line. Cultures were fed with different amounts of cystine in the feed medium. The results indicated that glutamine-independent NS0 cell lines are capable of producing humanized antibodies.

In summary, our studies showed that in an attempt to overcome glutamine dependence, the NS0 cell line was slowly adapted to glutamine-free conditions. The NS0 variant no longer requires supplemental glutamine in the medium for growth. A glutamine-independent cell line, which produces little to no ammonia during the course of a culture holds great promise as a foundation for future cell line development work. The adapted cell line is a suitable host for future production cell lines and cGMP manufacture.

The invention, and the manner and process of making and using it, are now described in such full, clear, concise, and exact terms as to enable any person skilled in the art to which it pertains, to make and use the same. It is to be understood that the foregoing describes preferred embodiments of the present invention and that modifications may be made therein without departing from the spirit or scope of the present invention as set forth in the claims to particularly point out and distinctly claim the subject matter regarded as invention, the following claims conclude this specification.

We claim:

1. A NS0 myeloma cell line capable of growing in a medium containing less than 5 mM glutamine.

2. The NS0 myeloma cell line according to claim 1, wherein said NS0 myeloma cell line is capable of growing in a glutamine-free medium.

3. The NS0 myeloma cell line according to claim 2, wherein said NS0 myeloma cell line is capable of growing in a serum-free medium.

4. The NS0 myeloma cell line according to claim 3, wherein said NS0 myeloma cell line is capable of growing in a cholesterol-free medium.

5. A NS0 myeloma cell line having the accession number ATCC PTA-4106.

6. A cell derived from a NS0 myeloma cell line according to claim 1.

7. A cell derived from a NS0 myeloma cell line according to claim 5.

8. The method of reducing toxicity of a cell culture comprising growing the cells derived from the NS0 myeloma cell line according to claim 2 in a glutamine free medium in said cell culture.

9. The method of making a recombinant polypeptide comprising:
(a) transfecting an expression vector comprising a DNA encoding the recombinant antibody into a cell of the NS0 myeloma cell line according to claim 1;
(b) culturing said transfected cell in a glutamine free medium; and
(c) secreting said recombinant polypeptide.

10. The method according to claim 9, wherein said recombinant polypeptide is a recombinant antibody.

11. The method according to claim 9, wherein said glutamine-free medium is cholesterol-free and serum-free.

12. The method of generating the NS0 myeloma cell line according to claim 2 comprising:
(a) culturing the NS0 cell line in a medium supplied with glutamine;
(b) reducing concentration of glutamine gradually until the medium contains no glutamine; and
(c) selecting NS0 cells that survive the medium containing no glutamine.
